(19) United States
(12) Patent Application Publication
(10) Pub. No.: US 2022/0151823 A1
(43) Pub. Date: May 19, 2022

(54) Vein Heating Assembly

(71) Applicant: Laverne Wright, Justice, IL (US)

(72) Inventor: Laverne Wright, Justice, IL (US)

(21) Appl. No.: 16/952,186

(22) Filed: Nov. 19, 2020

(51) Int. Cl. A61F 7/00; A61M 5/52

(52) U.S. Cl. CPC A61F 7/0053; A61M 5/52; A61M 2205/362; A61M 2209/084; A61F 2007/0093; A61M 2205/505; A61M 2205/8262; A61F 2007/0034; A61F 2007/0062; A61M 2205/3633

(57) ABSTRACT

A vein heating assembly includes a sleeve that has a first portion which is hingedly coupled to a second portion. The first portion is positionable in an open position to have a patient's arm positioned therein and the first portion is positionable in a closed position to surround the patient's arm. A plurality of nozzles is each integrated into the sleeve. A plurality of heating elements is each coupled to the sleeve to warm the patient's arm when the heating elements are turned on thereby increasing blood flow in the arm for enhancing the appearance of veins. A steam unit is integrated into the sleeve to produce steam that is delivered onto the patient's arm for increasing blood flow in the arm for enhancing the appearance of veins.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to heating devices and more particularly pertains to a new stimulation device for increasing the visibility of veins in a patient's arm for easing an intravenous injection.

### (2) Description of Related Art Including Information Disclosed Under 37 CFR 1.97 and 1.98

The prior art relates to heating devices including a fluid heating device for warming a thermal blanket. The prior art also discloses a variety of vein inflation devices for inflating veins which each employ a variation of an inflatable cuff. The prior art discloses a therapeutic heat pad that is heated with an external heat source.

### BRIEF SUMMARY OF THE INVENTION

An embodiment of the disclosure meets the needs presented above by generally comprising a sleeve that has a first portion which is hingedly coupled to a second portion. The first portion is positionable in an open position to have a patient's arm positioned therein and the first portion is positionable in a closed position to surround the patient's arm. A plurality of nozzles is each integrated into the sleeve. A plurality of heating elements is each coupled to the sleeve to warm the patient's arm when the heating elements are turned on thereby increasing blood flow in the arm for enhancing the appearance of veins. A steam unit is integrated into the sleeve to produce steam that is delivered onto the patient's arm for increasing blood flow in the arm for enhancing the appearance of veins.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a vein heating assembly according to an embodiment of the disclosure.

FIG. 2 is a right side view of an embodiment of the disclosure.

FIG. 3 is a left side view of an embodiment of the disclosure.

FIG. 4 is a front view of an embodiment of the disclosure.

FIG. 5 is a front view of an embodiment of the disclosure showing a sleeve in an open position.

FIG. 6 is a perspective view of a plurality of nozzles of an embodiment of the disclosure.

FIG. 7 is a cross sectional view taken along line 7-7 of FIG. 5 of an embodiment of the disclosure.

FIG. 8 is a schematic view of an embodiment of the disclosure.

### DETAILED DESCRIPTION OF THE INVENTION

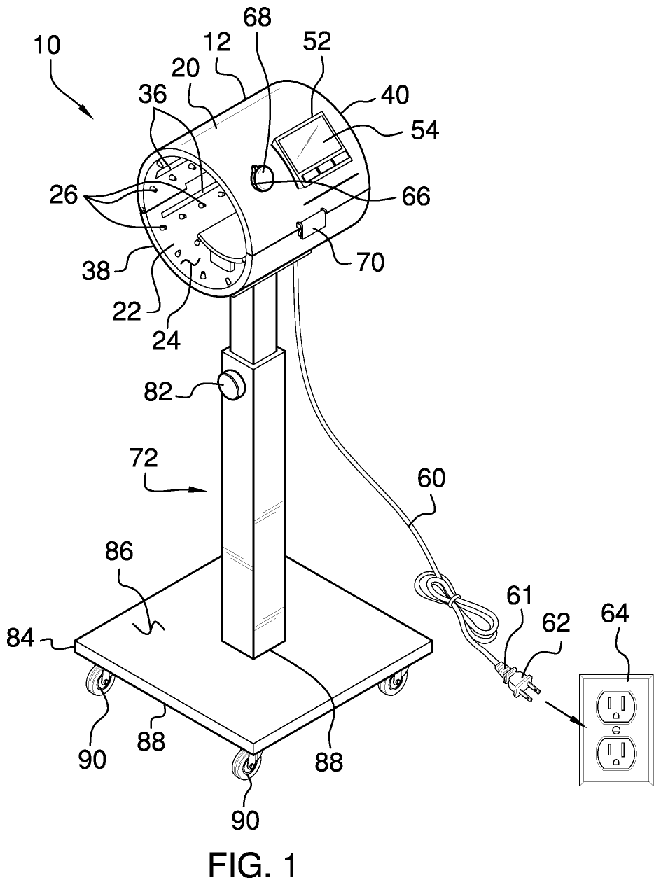

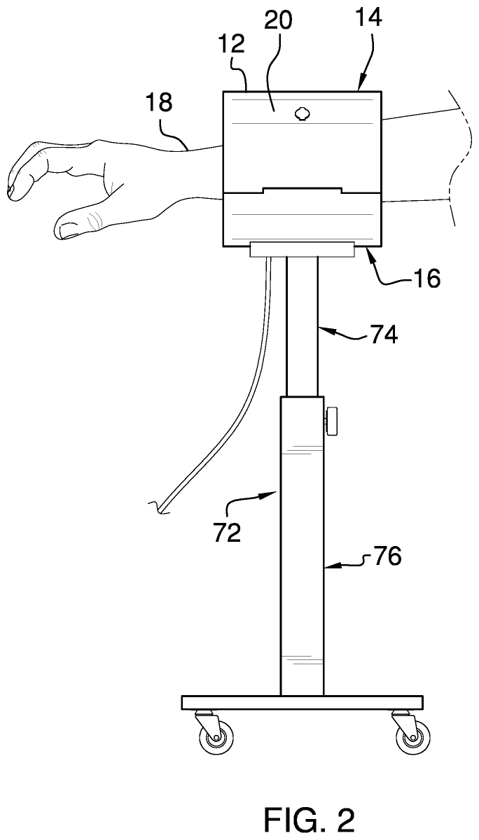

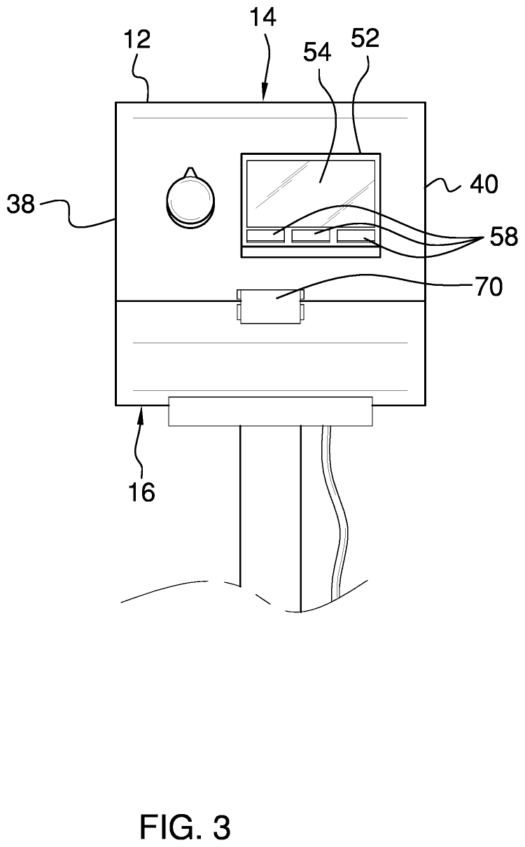

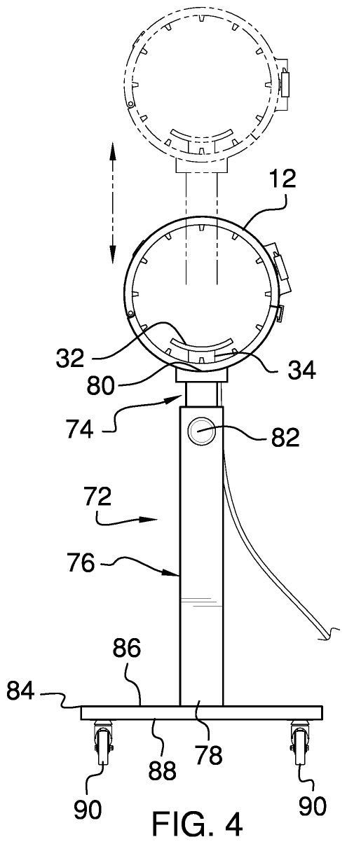

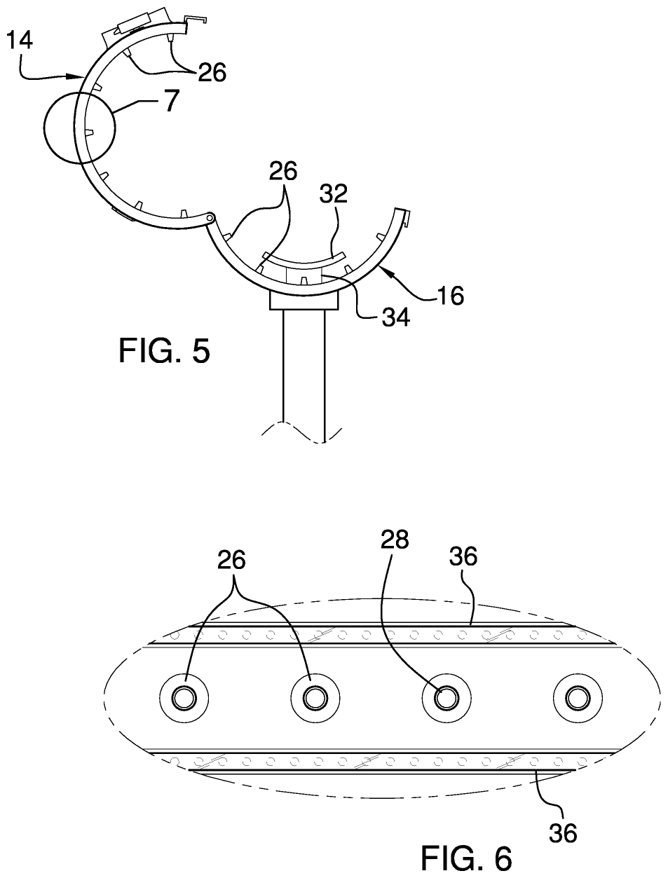

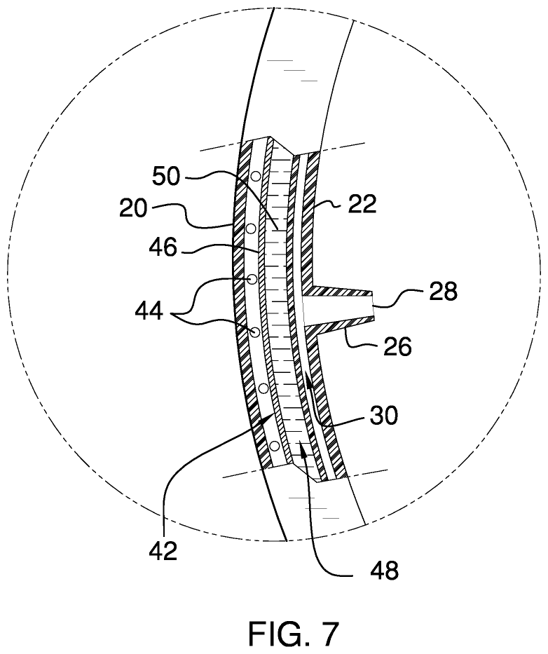

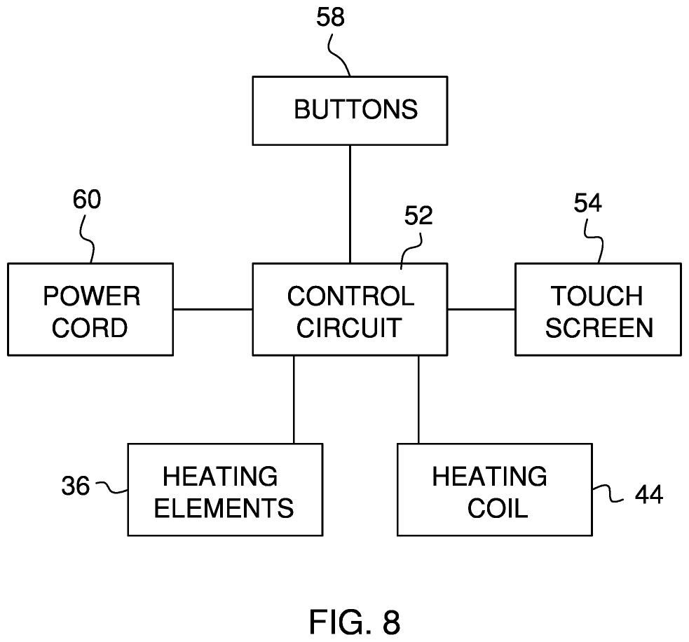

With reference now to the drawings, and in particular to FIGS. 1 through 8 thereof, a new stimulation device embodying the principles and concepts of an embodiment of the disclosure and generally designated by the reference numeral 10 will be described.

As best illustrated in FIGS. 1 through 8, the vein heating assembly 10 generally comprises a sleeve 12 that has a first portion 14 which is hingedly coupled to a second portion 16. The first portion 14 is positionable in an open position to have a patient's arm 18 positioned therein. Conversely, the first portion 14 is positionable in a closed position to surround the patient's arm 18. The sleeve 12 has an outer wall 20 and an inner wall 22, the sleeve 12 is hollow and the inner wall 22 has an outer surface 24. The inner wall 22 has a plurality of nozzles 26 each being integrated therein. The nozzles 26 extend away from the outer surface 24 of the inner wall 22 such that each of the nozzles 26 is directed toward the patient's arm 18 when the sleeve 12 surrounds the patient's arm 18.

Each of the nozzles 26 has a distal end 28 with respect to the outer surface 24 and the distal end 28 of each of the nozzles 26 is open. The nozzles 26 are spaced apart from each other and are distributed over an entirety of the outer surface 24. Each of the inner wall 22 and the outer wall 20 is comprised of a thermally insulating material. The inner wall 22 has a steam space 30 integrated therein and the steam space 30 is in fluid communication with each of the plurality of nozzles 26. An arm rest 32 is coupled to the sleeve 12 for supporting the patient's arm 18 when the patient's arm 18 is positioned in the sleeve 12. The arm rest 32 is concavely arcuate to conform to the patient's arm 18. The arm rest 32 includes a pair of supports 34 that is each coupled to the outer surface 24 of the inner wall 22 of the sleeve 12 thereby spacing the arm rest 32 from the outer surface 24.

A plurality of heating elements 36 is provided and each of the heating elements 36 is coupled to the sleeve 12. Each of the heating elements 36 warms the patient's arm 18 when the heating elements 36 are turned on thereby increasing blood flow in the arm 18 for enhancing the appearance of veins. Each of the heating elements 36 is positioned on the outer surface 24 of the inner wall 22 and each of the heating elements 36 is elongated to extend substantially between a front end 38 and a back end 40 of the sleeve 12. The heating elements 36 are spaced apart from each other and are distributed around a full circumference of the inner wall 22. Additionally, each of the heating elements 36 may comprise a light emitter that emits infra-red radiation for heating the patient's arm 18.

A steam unit 42 is integrated into the sleeve 12 for producing steam. The steam unit 42 is in fluid communication with each of the nozzles 26 to deliver the steam onto the patient's arm 18 for increasing blood flow in the arm 18 for enhancing the appearance of veins. The steam unit 42 comprises a heating coil 44 that is positioned between the inner wall 22 and the outer wall 20 of the sleeve 12. A heat shield 46 is positioned between the heating coil 44 and the inner wall 22 to define a water space 48 between the heat shield 46 and the inner wall 22 which can contain water 50. The heat shield 46 is comprised of a thermally conductive material thereby facilitating the heating coil 44 to heat the heat shield 46 when the heating coil 44 is turned on. In this way the heat shield 46 heats the water to produce steam. Additionally, the water space 48 is in fluid communication with the steam space 30 such that the steam space 30 can receive the steam from the steam space 30.

A control unit 52 is coupled to the sleeve 12 such that the control unit 52 is accessible to a user. The control unit 52 is in communication with each of the heating elements 36 and the steam unit 42. The control unit 52 comprises a touch screen 54 that is coupled to the outer wall 20 of the sleeve 12 and the touch screen 54 is positioned on the first portion 14. The touch screen 54 may comprise an LED or other type of electronic touch screen.

The control unit 52 includes a control circuit 56 that is integrated into the touch screen 54. The control circuit 56 is electrically coupled to the touch screen 54 and the control circuit 56 is electrically coupled to each of the heating elements 36 and the heating coil 44. The control unit 52 includes a plurality of buttons 58 that is each movably coupled to the outer wall 20 of the sleeve 12. Each of the buttons 58 is electrically coupled to the control circuit 56 and each of the buttons 58 controls operational parameters of respective ones of the heating elements 36 and the heating coil 44. A power cord 60 is coupled to and extends away from the sleeve 12. The power cord 60 is electrically coupled to the control circuit 56 and the power cord 60 has a distal end 61 with respect to the sleeve 12. A male plug 62 is electrically coupled to the distal end 28 and the male plug 62 is pluggable into a power source 64 comprising a female electrical outlet.

A fill port 66 is integrated into the outer wall 20 of the sleeve 12 and the fill port 66 is in fluid communication with the water space 48 for filling the water space 48 with water. The fill port 66 includes a closure 68 for opening and closing the fill port 66. A latch 70 is movably coupled to the first portion 14 of the sleeve 12 and the latch 70 releasably engages the second portion 16 for retaining the first portion 14 in the closed position. A stand 72 is coupled to the sleeve 12 and the stand 72 comprises a first section 74 slidably that engages a second section 76 such that the stand 72 has a telescopically adjustable height. The stand 72 has a lower end 78 and an upper end 80; the lower end 78 is associated with the first section 74 and the upper end 80 is associated with the second section 76. The upper end 80 is coupled to the outer wall 20 of the second portion 16 of the sleeve 12.

A screw 82 is rotatably coupled to the first section 74 of the stand 72 and the screw 82 engages the second section 76 when the screw 82 is tightened to retain the stand 72 at a selected height. The screw 82 disengages the second section 76 when the screw 82 is loosened to facilitate the stand 72 to be lengthened or shortened. A base 84 is included that has a top surface 86 and a bottom surface 88. The top surface 86 is coupled to the lower end 78 of the stand 72 and the stand 72 is centrally positioned on the base 84. A plurality of rollers 90 is each rotatably coupled to the bottom surface 88 of the base 84 to roll along a support surface.

In use, the patient's arm 18 is positioned in the sleeve 12 and each of the heating elements 36 and the steam unit 42 is turned on the heat the patient's arm 18. In this way blood flow in the patient's arm 18 is increased which subsequently increases the visibility of veins in the patient's arm 18. In this way an intravenous injection can more easily be performed on the patient's arm 18. The patient's arm 18 is removed from the sleeve 12 prior to administering the intravenous injection. Additionally, the patient's arm 18 is left in the sleeve 12 a sufficient duration of time to make the veins more visible.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A vein heating assembly for enlarging veins in a patient's arm to ease an intravenous injection, said assembly comprising:
a sleeve having a first portion being hingedly coupled to a second portion, said first portion being positionable in an open position wherein said sleeve is configured to have a patient's arm positioned therein, said first portion being positionable in a closed position wherein said sleeve is configured to surround the patient's arm, said sleeve having a plurality of nozzles each being integrated therein;
a plurality of heating elements, each of said heating elements being coupled to said sleeve wherein each of said heating elements is configured to warm the patient's arm when said heating elements are turned on thereby increasing blood flow in the arm for enhancing the appearance of veins;
a steam unit being integrated into said sleeve wherein said steam unit is configured to produce steam, said steam unit being in fluid communication with each of said nozzles wherein said steam unit is configured to deliver the steam onto the patient's arm for increasing blood flow in the arm for enhancing the appearance of veins;
a control unit being coupled to said sleeve wherein said control unit is configured to be accessible to a user, said control unit being in communication with each of said heating elements and said steam unit;
a stand being coupled to said sleeve, said stand comprising a first section slidably engaging a second section such that said stand has a telescopically adjustable height, said stand having a lower end and an upper end, said lower end being associated with said first section, said upper end being associated with said second section;
a base being coupled to said stand; and
a plurality of rollers, each of said rollers being rotatably coupled to said base wherein each of said roller is configured to roll along a support surface.

2. The assembly according to claim 1, wherein said sleeve has an outer wall and an inner wall, said sleeve being hollow, said inner wall having an outer surface, each of said nozzles being integrated into outer surface of said inner wall, said nozzles extending away from said outer surface of said inner wall wherein each of said nozzles is configured to be directed toward the patient's arm when said sleeve surrounds the patient's arm.

3. The assembly according to claim 2, wherein each of said nozzles has a distal end with respect to said outer surface, said distal end of each of said nozzles being open, said nozzles being spaced apart from each other and being distributed over an entirety of said outer surface.

4. The assembly according to claim 2, wherein each of said inner wall and said outer wall is comprised of a thermally insulating material.

5. The assembly according to claim 2, wherein said inner wall has a steam space being integrated therein, said steam space being in fluid communication with each of said plurality of nozzles.

6. The assembly according to claim 2, further comprising an arm rest being coupled to said sleeve wherein said arm rest is configured to support the patient's arm when the patient's arm is positioned in said sleeve, said arm rest being concavely arcuate wherein said arm rest is configured to conform to the patient's arm, said arm rest including pair of supports that is each coupled to said outer surface of said inner wall of said sleeve thereby spacing said arm rest from said outer surface.

7. The assembly according to claim 2, wherein each of said heating elements is positioned on said outer surface of said inner wall, each of said heating elements being elongated to extend substantially between a front end and a back end of said sleeve, said heating elements being spaced apart from each other and being distributed around a full circumference of said inner wall.

8. The assembly according to claim 2, wherein said steam unit comprises a heating coil being positioned between said inner wall and said outer wall of said sleeve.

9. The assembly according to claim 8, wherein:
said inner wall has a steam space being integrated therein, said steam space being in fluid communication with each of said plurality of nozzles; and
said steam unit includes a heat shield being positioned between said heating coil and said inner wall to define a water space between said heat shield and said inner wall wherein said water space is configured to contain water, said heat shield being comprised of a thermally conductive material thereby facilitating said heating coil to heat said heat shield when said heating coil is turned on wherein said heat shield is configured to heat the water to produce steam, said water space being in fluid communication with said steam space wherein said steam space is configured to receive the steam from said steam space.

10. The assembly according to claim 8, wherein said control unit comprises:
a touch screen being coupled to said outer wall of said sleeve, said touch screen being positioned on said first portion; and
a control circuit being integrated into said touch screen, said control circuit being electrically coupled to said touch screen, said control circuit being electrically coupled to each of said heating elements, said control circuit being electrically coupled to said heating coil.

11. The assembly according to claim 10, further comprising a plurality of buttons, each of said buttons being movably coupled to said outer wall of said sleeve, each of said buttons being electrically coupled to said control circuit, each of said buttons controlling operational parameters of respective ones of said heating elements and said heating coil.

12. The assembly according to claim 10, further comprising a power cord being coupled to and extending away from said sleeve, said power cord being electrically coupled to said control circuit, said power cord having a distal end with respect to said sleeve, said distal end having a male plug being electrically coupled thereto, said male plug being pluggable into a power source comprising a female electrical outlet.

13. The assembly according to claim 9, further comprising a fill port being integrated into said outer wall of said sleeve, said fill port being in fluid communication with said water space wherein said fill port is configured to fill the water space with water, said fill port including a closure for opening and closing said fill port.

14. The assembly according to claim 1, further comprising a latch being movably coupled to said first portion of said sleeve, said latch releasably engaging said second portion for retaining said first portion in said closed position.

15. The assembly according to claim 1, further comprising a screw being rotatably coupled to said first section of said stand, said screw engaging said second section when said screw is tightened to retain said stand at a selected height, said screw disengaging said second section when said screw is loosened to facilitate said stand to be lengthened or shortened.

16. A vein heating assembly for enlarging veins in a patient's arm to ease an intravenous injection, said assembly comprising:
a sleeve having a first portion being hingedly coupled to a second portion, said first portion being positionable in an open position wherein said sleeve is configured to have a patient's arm positioned therein, said first portion being positionable in a closed position wherein said sleeve is configured to surround the patient's arm, said sleeve having an outer wall and an inner wall, said sleeve being hollow, said inner wall having an outer surface, said inner wall having a plurality of nozzles each being integrated therein, said nozzles extending away from said outer surface of said inner wall wherein each of said nozzles is configured to be directed toward the patient's arm when said sleeve surrounds the patient's arm, each of said nozzles having a distal end with respect to said outer surface, said distal end of each of said nozzles being open, said nozzles being spaced apart from each other and being distributed over an entirety of said outer surface, each of said inner wall and said outer wall being comprised of a thermally insulating material, said inner wall having a steam space being integrated therein, said steam space being in fluid communication with each of said plurality of nozzles;
an arm rest being coupled to said sleeve wherein said arm rest is configured to support the patient's arm when the patient's arm is positioned in said sleeve, said arm rest being concavely arcuate wherein said arm rest is configured to conform to the patient's arm, said arm rest including pair of supports that is each coupled to said outer surface of said inner wall of said sleeve thereby spacing said arm rest from said outer surface;
a plurality of heating elements, each of said heating elements being coupled to said sleeve wherein each of said heating elements is configured to warm the patient's arm when said heating elements are turned on thereby increasing blood flow in the arm for enhancing the appearance of veins, each of said heating elements being positioned on said outer surface of said inner wall, each of said heating elements being elongated to extend substantially between a front end and a back end of said sleeve, said heating elements being spaced apart from each other and being distributed around a full circumference of said inner wall;
a steam unit being integrated into said sleeve wherein said steam unit is configured to produce steam, said steam unit being in fluid communication with each of said nozzles wherein said steam unit is configured to deliver the steam onto the patient's arm for increasing blood flow in the arm for enhancing the appearance of veins, said steam unit comprising:
a heating coil being positioned between said inner wall and said outer wall of said sleeve; and
a heat shield being positioned between said heating coil and said inner wall to define a water space between said heat shield and said inner wall wherein said water space is configured to contain water, said heat shield being comprised of a thermally conductive material thereby facilitating said heating coil to heat said heat shield when said heating coil is turned on wherein said heat shield is configured to heat the water to produce steam, said water space being in fluid communication with said steam space wherein said steam space is configured to receive the steam from said steam space;
a control unit being coupled to said sleeve wherein said control unit is configured to be accessible to a user, said control unit being in communication with each of said heating elements and said steam unit, said control unit comprising:
a touch screen being coupled to said outer wall of said sleeve, said touch screen being positioned on said first portion;
a control circuit being integrated into said touch screen, said control circuit being electrically coupled to said touch screen, said control circuit being electrically coupled to each of said heating elements, said control circuit being electrically coupled to said heating coil;
a plurality of buttons, each of said buttons being movably coupled to said outer wall of said sleeve, each of said buttons being electrically coupled to said control circuit, each of said buttons controlling operational parameters of respective ones of said heating elements and said heating coil; and
a power cord being coupled to and extending away from said sleeve, said power cord being electrically coupled to said control circuit, said power cord having a distal end with respect to said sleeve, said distal end having a male plug being electrically coupled thereto, said male plug being pluggable into a power source comprising a female electrical outlet;
a fill port being integrated into said outer wall of said sleeve, said fill port being in fluid communication with said water space wherein said fill port is configured to fill the water space with water, said fill port including a closure for opening and closing said fill port;
a latch being movably coupled to said first portion of said sleeve, said latch releasably engaging said second portion for retaining said first portion in said closed position;
a stand being coupled to said sleeve, said stand comprising a first section slidably engaging a second section such that said stand has a telescopically adjustable height, said stand having a lower end and an upper end, said lower end being associated with said first section, said upper end being associated with said second section;
a screw being rotatably coupled to said first section of said stand, said screw engaging said second section when said screw is tightened to retain said stand at a selected height, said screw disengaging said second section when said screw is loosened to facilitate said stand to be lengthened or shortened;
a base having a top surface and a bottom surface, said top surface being coupled to said lower end of said stand, said stand being centrally positioned on said base; and
a plurality of rollers, each of said rollers being rotatably coupled to said bottom surface of said base wherein each of said roller is configured to roll along a support surface.
